(19) United States
(12) Patent Application Publication
(10) Pub. No.: US 2007/0293816 A1
(43) Pub. Date: Dec. 20, 2007

(54) Microprojection Array Application with Grouped Microprojections for High Drug Loading

(75) Inventors: Keith Chan, Brookline, MA (US); Rajan Patel, Menlo Park, CA (US); Peter E. Daddona, Menlo Park, CA (US); Cedric Wright, Mountain View, CA (US); Neha Agarwal, Los Altos Hills, CA (US)

Correspondence Address: WILSON SONSINI GOODRICH & ROSATI & MACROFLUX CORP., 650 PAGE MILL ROAD, PALO ALTO, CA, 94304, US

(73) Assignee: ALZA CORPORATION, Mountain View, CA (US)

(21) Appl. No.: 11/740,235

(22) Filed: Apr. 25, 2007

Related U.S. Application Data
(60) Provisional application No. 60/794,941, filed on Apr. 25, 2006.

(51) Int. Cl. A61M 37/00

(52) U.S. Cl. 604/46

(57) ABSTRACT

A transdermal drug delivery system with microprojections for disrupting a body surface to an individual. At least some of the microprojections form groups in a microprojection array. There are repeated units of such groups in the microprojection array.

### CROSS-REFERENCE

This application claims the benefit of U.S. Provisional Application No. 60/794,941, filed Apr. 25, 2006, which application is incorporated herein by reference.

### BACKGROUND OF THE INVENTION

This invention relates to an apparatus and method for applying a microprojection array to the stratum corneum by impact, and more particularly, the invention relates to a microprojection array having high drug loading thereon.

The natural barrier function of the body surface, such as skin, presents a challenge to delivery therapeutics into circulation. Transdermal devices for the delivery of biologically active agents or drugs have been used for maintaining health and therapeutically treating a wide variety of ailments. For example, analgesics, steroids, etc., have been delivered with such devices. Transdermal drug delivery can generally be considered to belong to one of two groups: transport by a “passive” mechanism or by an “active” transport mechanism. In the former, such as drug delivery skin patches, the drug is incorporated in a solid matrix, a reservoir, and/or an adhesive system.

There are various ways to increase transdermal delivery rates. One way to increase the transdermal delivery of agents is to pretreat the skin with, or co-deliver with the beneficial agent, a skin permeation enhancer. A permeation enhancer substance, when applied to a body surface through which the agent is delivered, enhances the transdermal flux of the agent such as by increasing the permselectivity and/or permeability of the body surface, and/or reducing the degradation of the agent.

Another type of transdermal drug delivery is active transport in which the drug flux is driven by various forms of energy. Iontophoresis, for example, is an “active” electrotransport delivery technique that transports solubilized drugs across the skin by an electrical current. The feasibility of this mechanism is constrained by the solubility, diffusion and stability of the drugs, as well as electrochemistry in the device. The transport of the agent is induced or enhanced by the application of an applied electrical potential, which results in the application of electric current, to deliver or enhance delivery of the agent.

However, at the present many drugs and pharmaceutical agents still cannot be efficiently delivered by conventional passive patches or electrotransport systems through intact body surfaces. There is an interest in the percutaneous or transdermal delivery of larger molecules such as peptides and proteins to the human body as increasing number of medically useful peptides and proteins become available in large quantities and pure form. The transdermal delivery of larger molecules such as peptides and proteins still faces significant challenges. In many instances, the rate of delivery or flux of large molecules, such as polypeptides, through the skin is insufficient to produce a desired therapeutic effect due to their large size and molecular weight. In addition, polypeptides, proteins, and many biologics are easily degraded during and after penetration into the skin, prior to reaching target cells. On the other hand, the passive transdermal flux of many low molecular weight compounds is too limited to be therapeutically effective.

Yet another method to increase transdermal flux (e.g., across skin) is to mechanically penetrate or disrupt the skin. This technique has been mentioned in, for example, U.S. Pat. No. 5,879,326 issued to Godshall, et al., U.S. Pat. No. 3,814,097 issued to Ganderton, et al., U.S. Pat. No. 5,279,544 issued to Gross, et al., U.S. Pat. No. 5,250,023 issued to Lee, et al., U.S. Pat. No. 3,964,482 issued to Gerstel, et al., Reissue 25,637 issued to Kravitz, et al., and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365. These devices use piercing elements or microprojections of various shapes and sizes to pierce the outermost layer (i.e., the stratum corneum) of the skin. The microprojections disclosed in these references generally extend perpendicularly from a thin, flat member, such as a pad or sheet. The microprojections in some of these devices are extremely small, some having dimensions (i.e., a microblade length and width) of only about 25-400μ and a microblade thickness of only about 5-50μ. Other penetrating elements are hollow needles having diameters of about 10μ or less and lengths of about 50-100μ. These tiny stratum corneum piercing/cutting elements are meant to make correspondingly small microslits/microcuts in the stratum corneum for enhanced transdermal agent delivery or transdermal body analyte sampling therethrough. The perforated skin provides improved flux for sustained agent delivery or sampling through the skin. In many instances, the microslits/microcuts in the stratum corneum have a length of less than 150μ and a width that is substantially smaller than their length.

When microprojection arrays are used to improve delivery or sampling of agents through the skin, consistent, complete, and repeatable microprojection penetration is desired. Microprojection arrays generally have the form of a thin, flat pad or sheet with a plurality of microprojections extending roughly perpendicularly upward and are difficult to handle if they are too big. When an individual manually pushes the microprotrusion array on the skin by hand, the push force may be hard to control and may be uneven across the area of the array. Thus, mechanically actuated devices have been invented to apply a microprojection array to the stratum to effect microprojection skin piercing penetration in a more consistent and repeatable manner. However, even with the help of a mechanical actuator, a large microprojection array is still hard to apply to the body surface since body surfaces are generally not actually flat. Further, large microprojection arrays are inconvenient and uncomfortable for the patient. Because many chemical drugs are not highly potent, to deliver an effective amount of the drug, increasing the drug loading per unit planar area of a microprojection member holding the microprojection array is desirable. The ability to increase drug loading on the device can be critical for patient compliance and the successful application of such a device.

Typically, a drug coating for microprojection array is formed on each microprojection by wetting the microprojection with a drug formulation as it dips into a drug formulation film. The repeated dipping increases the total drug loading on each tip. However, repeated dipping increases the drug coating profile and the increasing drug coating profile not only hinders skin penetration but also increases the force imparted on the drug coating during skin penetration, thereby increasing the risk of the drug coating sloughing off prior to delivery.

What is needed is a microprojection array that has a higher capacity to hold drug than prior devices. The present invention provides systems and methods of making and using such systems in which the microprojection array has microprojection groupings to increase drug loading.

### SUMMARY OF THE INVENTION

This invention is related to microprojection systems and methodology that provides a microprojection array for application of the microprojections to the stratum corneum. The microprojection array includes a plurality of microprojections that penetrate the stratum corneum to improve transport of an agent across the stratum corneum. At least some of the microprojections are positioned in groups. Preferably, a drug coating is coated on at least a portion of the microprojections in the group.

In accordance with an additional aspect of the invention, in a device for drug delivery is a microprojection array with a plurality of stratum corneum piercing microprojections for piercing stratum corneum. At least some of the microprojections are positioned in groups. In some aspects the microprojections are arranged in groups of at least two adjacent microprojections. In further aspect of the invention, a group of microprojections can consist of blade shaped microprojections with a sharp cutting point. In another aspect of the invention, the groups of microprojections can consist of microprojections that have surfaces that face surfaces of adjacent microprojections in the group. In an additional aspect, the microprojections are positioned in groups and at least some of the microprojections have shafts of different length.

In accordance with an additional aspect of the invention, a device for drug delivery includes a microprojection array with a plurality of stratum corneum piercing microprojections for piercing stratum corneum. At least some of the microprojections are positioned in groups and in a group at least one microprojection leans towards another microprojection. Alternatively, the tips of the microprojections in the pair of microprojections can be oriented such that they are substantially parallel relative to each other.

In accordance with an additional aspect of the invention, a device for drug delivery includes a microprojection array with a plurality of stratum corneum piercing microprojections for piercing stratum corneum. Each microprojection of the microprojection array can have a base. At least some of the microprojections are positioned in groups and the base of each microprojection are spaced apart by less than 200 μm or by 10 μm to 100 μm.

In accordance with another aspect of the invention, a device for drug delivery includes a microprojection array with a plurality of stratum corneum piercing microprojections for piercing stratum corneum. At least some of the microprojections are positioned in groups and in a group a continuous drug coating bridges the microprojections of the group.

In accordance with another aspect of the invention, a device for drug delivery includes a microprojection array with a plurality of stratum corneum piercing microprojections for piercing stratum corneum. At least some of the microprojections are positioned in groups and in a group at least one microprojection leans towards another microprojection and a continuous drug coating bridges the microprojections of the group.

In accordance with another aspect of the invention, a device for drug delivery includes a microprojection array with a plurality of stratum corneum piercing microprojections for piercing stratum corneum. Each microprojection has a top portion extending out of a plane on the microprojection member and in at least some of the groups the microprojections have base portions extending more along a plane of the microprojection member than the top portions do and towards other microprojection portions in the group. In an alternative embodiment, the microprojections have base portions extending more along a plane of the microprojection member and away from other microprojections in the group than the top portions do.

In accordance with another aspect of the invention, a device for drug delivery includes a microprojection array with a plurality of stratum corneum piercing microprojections for piercing stratum corneum. At least some of the microprojections are positioned in groups. In at least some of the groups the microprojections have shafts of different lengths, a longer microprojection leaning to a shorter microprojection forming a pinnacle. In one aspect, the pinnacle formed between the microprojections can have an angle between 10 degrees and 60 degrees. In at least some of the groups a continuous drug coating coats at least top portions of the microprojections in a group, the drug coating having a meniscus bridging the microprojections in the group.

In another aspect, the present invention further provides a method of making a device with microprojections to pierce stratum corneum to facilitate drug delivery by forming at least some of the microprojections in groups. Preferably a drug coating is coated on at least some of the microprojections. Various shapes and configurations, materials of construction and drug coating parameters can be selected to result in the desired microprojection drug delivery device. In another aspect, the invention further provides a method for applying a stratum-corneum piercing drug deliver device by a microprojection device where the microprojections are arranged in groups. The microprojections may further comprise blade shaped microprojection pair that are facing each other and coated with a drug on at least a portion of the microprojections.

The grouping of microprojections in close proximity allows the microprojections to act as a “planar capillary” (e.g., a parallel plane capillary) and to shield and protected the drug coating therebetween from the impact forces during skin penetration, allowing the drug coating to penetrate deeper into the skin for effective drug delivery without coming off by the impact. In certain groups enabled by the present invention, adjacent microprojections converge in a way such that it facilitates skin penetration.

The inclusion of two or more microprojections into a group in the device with stratum corneum piercing microprojections facilitates better penetration of the microprojection through the stratum corneum and increases the drug loading with similar size of planar area in microprojection array. The grouping of microprojection increases the capacity of the microprojection to capture drug material on the microprojection, whereas otherwise a larger device with a larger volume and larger planar surface area would be required. This advantage provided by increased drug loading without increasing planar area is especially important for drugs that are less potent. Because large devices for piercing the stratum corneum are hard to handle and increase discomfort to the patient, the ability to increase drug loading on the device can be critical for patient compliance and the successful application of such devices. Thus, the present invention provides substantial benefits for drug delivery not available in the past.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The present invention is illustrated by way of example in embodiments and not limitation in the figures of the accompanying drawings in which like references indicate similar elements. The figures are not shown to scale unless indicated otherwise in the content. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a sectional view of an applicator device and microprojection array system according to the present invention.

FIG. 2 illustrates an isometric view in portion of a microprojection array member according to the present invention.

FIG. 3 illustrates a sectional view in portion of an embodiment of a group of microprojections according to the present invention.

FIG. 4 illustrates an isometric view in portion of another embodiment of a group of microprojections having a drug coating according to the present invention.

FIG. 5 illustrates an isometric view in portion of yet another embodiment of a group of microprojections forming a pinnacle according to the present invention.

FIG. 6 illustrates a sectional side view in portion of another embodiment of a group of microprojections according to the present invention.

FIG. 7 illustrates a sectional side view in portion of another embodiment of a group of microprojections according to the present invention.

FIG. 8 illustrates a sectional side view in portion of another embodiment of a group of microprojections with a drug coating with meniscus according to the present invention.

FIG. 9 illustrates an isometric view in portion of an embodiment of a group of microprojections according to the present invention.

FIG. 10 illustrates an isometric view in portion of another embodiment of a group of microprojections according to the present invention.

FIG. 11 illustrates an isometric view in portion of yet another embodiment of a group of microprojections according to the present invention.

FIG. 12 illustrates an isometric view in portion of yet another embodiment of a group of microprojections showing microprojection layers according to the present invention.

FIG. 13 illustrates an isometric in portion of yet another embodiment of a group of microprojections showing microprojection layers according to the present invention.

FIG. 14 illustrates an isometric in portion of yet another embodiment of a group of microprojections showing microprojection layers according to the present invention.

FIG. 15 shows a scanning electromicrograph of a microprojection array having microprojection pairs with drug coating.

FIG. 16 is a graph showing the drug content of microprojection member with paired microprojections compared to that of microprojection member without paired microprojections.

FIG. 17 is a scanning electromicrograph showing a portion of a microprojection array having pinnacle microprojection pairs with drug coating.

FIG. 18 is a scanning electromicrograph of an embodiment showing a portion of a microprojection array showing pairs of parallel microprojections with drug coating.

FIG. 19 is a scanning electromicrograph of another embodiment showing a portion of a microprojection array showing pairs of parallel microprojections with drug coating.

FIG. 20 illustrates a plan view in portion of yet another embodiment of a design of groups of microprojections according to the present invention.

FIG. 21 illustrates a plan view in portion of yet another embodiment of a design of groups of microprojections according to the present invention.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present invention relates to methods and devices for transdermal delivery of drugs using a microprojection device in which a microprojection array has groups of microprojections to facilitate penetration of the stratum corneum and/or to increase the surface area for holding drugs. For example, the proximity of the microprojections in the group can allow the more drug coating material to be held by the microprojections than otherwise possible.

In describing the present invention, the following terms will be employed, and are defined as indicated below. As used in this specification and the appended claims, the singular forms “a,” “an” and “the” include plural references unless the content clearly dictates otherwise.

As used herein, the term “transdermal” refers to the use of skin, mucosa, and/or other body surfaces as a portal for the administration of drugs by topical application of the drug thereto for passage into the systemic circulation. As described herein, the stratum corneum can be disrupted in such transdermal drug transport.

“Biologically active agent” is to be construed in its broadest sense to mean any material that is intended to produce some biological, beneficial, therapeutic, or other intended effect, such as enhancing permeation or relief of pain. As used herein, the term “drug” refers to any material that is intended to produce some biological, beneficial, therapeutic, or other intended effect, such as relief of pain, but not agents (such as permeation enhancers) the primary effect of which is to aid in the delivery of another biologically active agent such as the therapeutic agent transdermally.

As used herein, the term “therapeutically effective” refers to the amount of drug or the rate of drug administration needed to produce the desired therapeutic result.

The terms “microprojections” and “microprotrusions”, as used herein, refer to piercing elements that are adapted to pierce or cut through the stratum corneum into the underlying epidermis layer, or epidermis and dermis layers, of the skin of a living animal, particularly a mammal and more particularly a human.

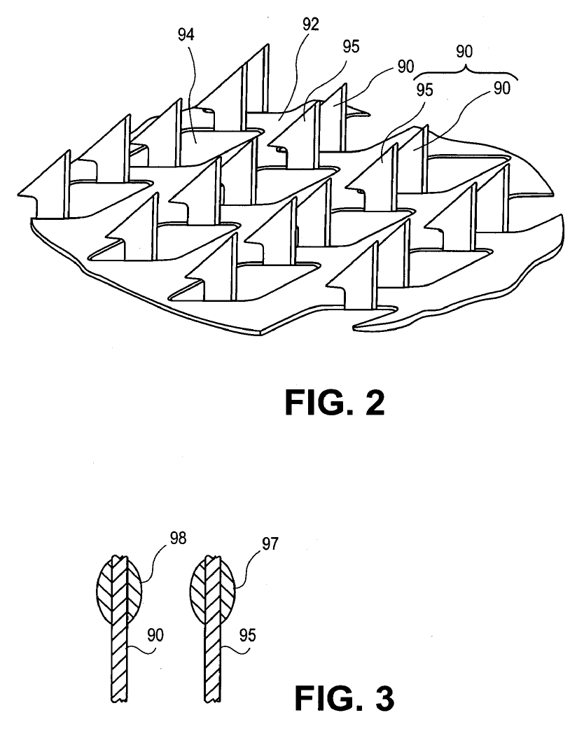

The term “microprojection array” or “microprotrusion array”, as used herein, refers to a plurality of microprojections arranged in an array for piercing the stratum corneum. The microprojection array may be formed by etching or punching a plurality of microprojections from a thin sheet or sheets and folding or bending the microprojections out of the plane of the sheet to form a configuration, such as the bent microprojections shown in FIG. 2. Such methods of making microprojections are known in the art. For example, U.S. Pat. Nos. 5,879,326; 6,050,988; 6,091,975; 6,537,264 and US Patent Publication 20040094503 disclose processes for making microprojections by etching substrates. Silicon and plastic microprojection members are described in U.S. Pat. No. 5,879,326. The microprojection array can also be formed by other known methods, such as by forming one or more strips having microprojections along an edge of each of the strip(s) as disclosed in U.S. Pat. No. 6,050,988. These patent publications are incorporated herein by reference in their entireties.

The term “group” when referred to microprojection arrangement means a plurality, e.g., two (a pair), or more, of neighboring microprojections that are closer to one another than to other microprojections. In many cases, there are repeating units of such groups of microprojections in the microprojection array.

The present invention involve devices and methodology that provide better penetration of the stratum corneum and/or an increased drug loading per unit size or planar surface area of a microprojection member having a microprojection array for piercing the stratum corneum. Through grouping microprojections in close proximity, such advantages over prior devices can be realized. For example, the microprojections in a group can have a continuous drug coating that bridges the microprojections.

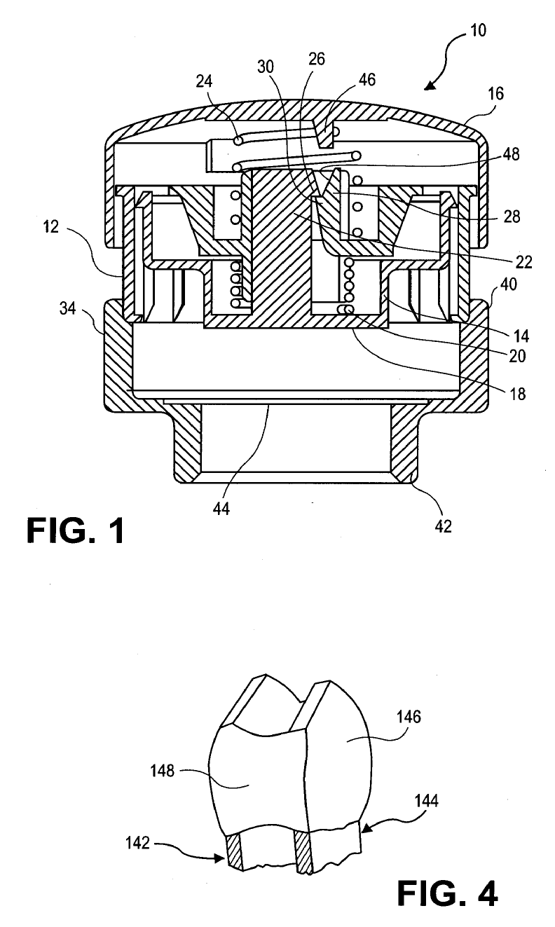

An applicator system can be used for applying a microprojection member as described below. Such a system includes an impact applicator for applying the microprojection member to the stratum corneum. The microprojection member can include a microprojection array. FIG. 1 shows a schematic sectional view of an exemplary microprojection device having an applicator, retainer, and microprojection array. Similar devices with actuators and retainers are described in United States patent documents 20020123675, 20050096586, 20050138926, 20050226922, and 20050089554, which are incorporated by reference herein. It is to be understood that such devices of these documents and other prior microprojection devices can be adapted to be used with the present invention. FIG. 1 illustrates an exemplary embodiment of an applicator 10 for use with a retainer 34 containing microprojection member 44.

However, the device of FIG. 1 is just an example and other applicator configurations may also be used with the microprojection arrays described herein. The applicator 10 includes a body 12 and a piston 14 movable within the body. A cap 16 is provided on the body 12 for activating the applicator to impact the stratum corneum with the microprojection member 44. An impact spring 20 is positioned around a post 22 of the piston 14 and biases the piston downward (i.e., towards the skin) with respect to the body 12. The piston 14 has an impact surface 18 that is substantially planar, slightly convex, or configured to match the contours of a particular body surface. The surface 18 of the piston 14 impacts the microprojection member 44 against the skin causing the microprojections 90 to pierce the stratum corneum of, for example, the skin of a patient.

FIG. 1 shows the piston 14 in a cocked position. When the applicator is cocked, the piston 14 is pressed up inside the body 12 and locked in place by a locking mechanism. The locking mechanism includes a stop catch 26 on the post 22 and a flexible finger 28 on the body 12 having a corresponding latch stop 30. As the piston 14 is moved toward the body 12 compressing the impact spring 20, the stop catch 26 flexes the finger 28 and snaps over the corresponding latch stop 30 of the flexible finger. The cocking step is performed by a single compression motion that both cocks and locks the piston 14 in the cocked position.

In the cocked position, catch 26 and latch 30 on the piston 14 and body 12 are releasably engaged, preventing downward motion of the piston in the body. FIG. 1 also illustrates the patch retainer 34 mounted on the body 12. The activation of the applicator 10 by the release of the locking mechanism is performed by downward force applied to the applicator cap 16 while the end 42 of the applicator is held against the skin. The cap 16 is biased in a direction away from the skin by a hold down spring 24 that is positioned between the body 12 and the cap. The cap 16 includes a pin 46 extending downward from the cap. When the cap 16 is pressed downward against the bias of the hold down spring 24, the pin 46 contacts ramp 48 on flexible finger 28 moving the flexible finger outward and disengaging latch 30 of the flexible finger 28 from catch 26. This releases piston 14 and the piston moves downward impacting the stratum corneum with the microprojection member 44. The impact is applied substantially parallel to a central axis of the microprojection member 44. Preferably, the microprojection member is connected to the retainer by at least one frangible element (not shown in the figure) that is broken when the impact applicator is activated.

FIG. 2 illustrates an exemplary embodiment of a microprojection member having a microprojection array of the present invention. FIG. 2 shows a plurality of microprojections (or microprotrusions) in the form of microblades 90, which have a blade shape with a cutting sharp point. The microblades 90 extend at a substantially 90° angle from a sheet 92 having openings 94. The microprojections are preferably sized and shaped to penetrate the stratum corneum of the epidermis when pressure is applied to the microprojection member, for example, forming microslits on the body surface. The sheet 92 may be incorporated in an agent delivery patch or an agent-sampling patch that includes an agent (i.e., a pharmaceutical agent or drug) reservoir and/or an adhesive for attaching the patch to the stratum corneum.

It is preferred that at least some of the microprojections are arranged into groups. For example, as shown in FIG. 2, microprojection 90 and microprojection 95 are proximate to each other and form groups 96. In the group 96, the microprojections, e.g., microprojection 95 and microprojection 96 are closer to one another than to other microprojections that are not in the group. One of the advantages of grouping microprojections together is that they can penetrate the stratum corneum easier. Since skin is supple and flexible, when a sharp object is pressed onto the skin, it pushes the skin inward but does not immediately penetrate. This is analogous to a pencil point pushing against the skin will cause it to dimple but the skin does not allow the pencil point to break through the skin surface. Having a group (e.g., two) of microprojections close together will allow the skin to be taut between the microprojections in the group when the microprojections are pressed against the skin and therefore allow easier penetration. This will be particularly useful if the microprojections are relatively short and may not be able to penetrate adequately otherwise. Preferably a number of such groups are present as repeated units in the microprojection array.

Preferably the microprojections each have a drug coating with a drug (for example, on or near the tip of the microprojections). The microprojection member and microprojection array can be made with technology known in the art. Examples of agent delivery and sampling patches that incorporate a microprojection array are found in US20020016562, U.S. Pat. No. 6,537,264, WO 97/48440, WO 97/48441, WO 97/48442, the disclosures of which are incorporated herein by reference in their entireties. The microprojection array of FIG. 2 without a drug reservoir or a drug coating may also be applied alone as a skin pretreatment. In one embodiment of the invention, the microprojections have projection length of less than 1000 microns (μm). In a further embodiment, the microprojections have a projection length of less than 500 microns (μm), more preferably, less than about 250 μm. In some embodiments, the microprojections preferably have a normally extending portion of about 25 μm to 400 μm long, more preferably about 50 μm to 250 μm long. As used herein, “normally extending” means extending at an angle from the plane of a microprojection member and, although possible, need not be exactly 90°.

The microprojections can be formed from metallic materials such as titanium, stainless steel, and polymers. Techniques for making microprojection array (e.g., by etching) from such materials are known in the art. Generally, substrates for forming microprojections are about 3 microns (μm) to 50 μm thick, preferably about 15 μm to 35 μm thick. The microprojections typically have a width of about 5 μm to 250 μm, preferably about 100 μm to 150 μm. The thicknesses of the microprojections are about 3 μm to 50 μm, preferably about 10 μm to 30 μm. The microprojections may be formed in different shapes, such as needles, blades, pins, punches, and combinations thereof. If the microprojections are from the same sheet of material (for example, all were chemically etched from the same single sheet of titanium), the microprojection density is approximately 10 microprojections/cm², more preferably, in the range of approximately 200-5000 microprojections/cm². The distance between neighboring microprojections in a group can be about less than about 500 μm, preferably less than about 200 μm, more preferably about 10 μm to 160 μm, even more preferably about 10 μm to 100 μm, even more preferably about 50 μm to 100 μm, at the base of the microprojections. Typically the microprojections extend from a base plate upward. The distances are generally measured between the base positions of the upwardly extending portions. There can be openings near the microprojections on the microprojection member. Such openings can allow agents or drugs to pass if agents or drugs are placed under or in such openings. The number of openings per unit area through which the active agent (drug) passes is preferably from approximately 10 openings/cm² to about 2000 openings/cm².

As mentioned before, microprojections can have a drug coating to carry the drug to be delivered. FIG. 3 shows an embodiment of a group (e.g., a pair) of neighboring microprojections 90, 95 having drug coatings 97, 98 at the distal portions (or top portions) thereof. As used herein, “distal” means a direction that is towards the skin surface on which the microprojection is to be applied. In FIG. 3 the microprojections are substantially parallel to each other and the drug coatings 97, 98 from the two microprojections 95, 96 do not touch. The two microprojection can have the same drug formulation of have different drug formulations. For example, one microprojection can be coated with a formulation that exhibits a fast therapeutic onset, while the other registered can be coated with a formulation that exhibits a sustained therapeutic effect. Further, the microprojections can have different dosage.

A way to increase drug loading is to increase the amount of drug coating on a microprojection, as already mentioned. A further way to increase drug loading is to group neighboring microprojections close enough together to capture a continuous drug coating between the microprojections in the group. Thus, arranging the microprojections into a group will increase the volume of drug coating that can be held than otherwise possible. FIG. 4 illustrates an embodiment of a group (which in this case is a pair) of microprojections 142, 144. The microprojections 142, 144 extend in an about parallel fashion. A continuous drug coating 146 coats and extends from one microprojection 142 near its top to the other microprojection 144, forming a drug coating bridge 148. Thus, drug coating material bridges the microprojection 142, 144 and is sandwiched therebetween. Also, the drug coating material of the drug coating bridge 148 actually is continuous over and envelops the top portion of the microprojections 142, 144.

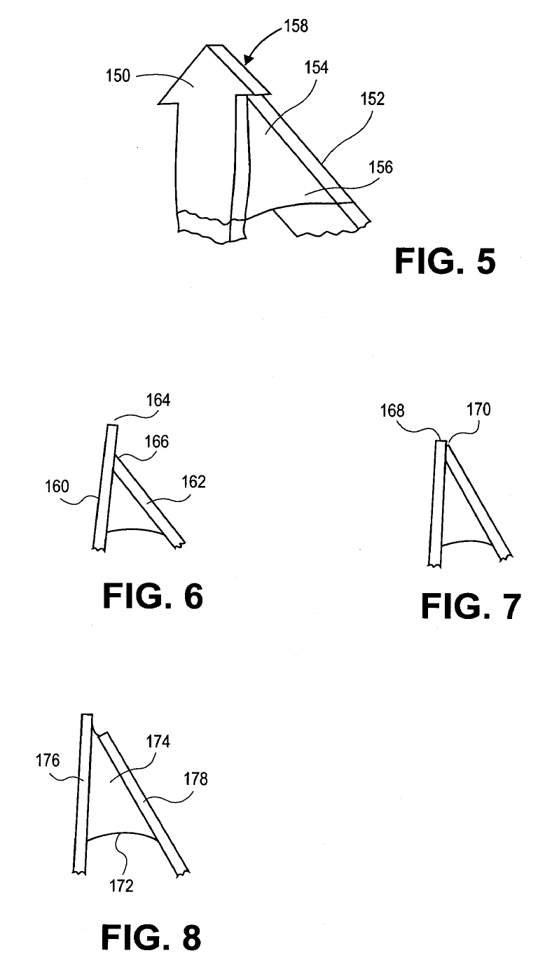

FIG. 5 shows an illustration of another alternative with a group (here a pair) of microprojections converging at the tips. In the embodiment of FIG. 5, microprojection 150 extends substantially straight up from the microprojection member planar plate (not shown) and microprojection 152 leans at an angle toward microprojection 150 so that the drug coating 154 forms a continuous bridge 156 coating the top portions of both of the microprojections. In this embodiment, microprojection 150 has an arrowhead shaped top portion. The converging of microblades forms a pinnacle 158 that can facilitate penetration of the stratum corneum. The angle of leaning (relative to the plane of the microprojection member) preferably is about 60° to slightly less than 90°, more preferably about 70° to 80°. The leaning microprojection can be longer, the same length or shorter than the one that is not leaning. Furthermore, one, two or more of the microblades in the group can be leaning.

The microblades can converge such that their tips are close together but not exactly touching. Alternatively, the microblades can converge to touch at the tips. Further, as shown in FIG. 6, one microblade (say, a first microblade) 160 can intercept a second microblade 162 along by the elongated portion of the first microblade 160 such that tip 164 of the first microblade 160 extends past the tip 166 and the body of the second microblade 162 (but not the other way around). The tip 166 of the second microprojection 162, although touching the first microprojection 160 in this embodiment, does not extend past the first microprojection. This way, during penetration of the stratum corneum, the tip 164 of first microblade 160 will initiate the penetration: Alternatively, the microblades can converge such that their tips 168, 170 are about even, as shown in FIG. 7. This way, the tips 168, 170 of the microblades generally penetrate the stratum corneum at about the same time.

The proximity of microprojections in a group allows the drug coating liquid before solidifying to be drawn and held by capillary action among the microprojections in a group. This is especially useful in embodiments with converging top portions because the capillary action tends to draw the liquid drug coating towards the tips of the microprojections, and therefore at a position suitable to delivery drug deeper into the skin. This phenomenon is especially evident in instances in which hydrophilic drug coating is coating hydrophilic microprojections, wherein there is a small contact angle for the liquid on a surface. Wetability of a liquid on a surface is related to the contact angle θ formed by the liquid-solid and the liquid-gas interfaces. If θ is greater than 90° the liquid tends to form droplets on the surface, i.e., the liquid does not wet the surface well. If θ is less than 90° the liquid tends to spread out over the surface. When the liquid forms a thin film on the surface i.e., wetting it well, θ tends to near zero. In instances of hydrophilic liquid on a hydrophilic surface, for example, as shown in FIG. 8, a concave shaped meniscus 172 would be formed by the capillary force in the drug coating 174 on the top portion of microprojections 176, 178 in a group. As used herein, even after the drug coating has solidified, the concaved shaped curve 172 is still called a meniscus for the sake of consistency. In FIG. 8, the tips of the microprojections 176 178 do not actually touch. However, the drug coating 172, due to its viscosity before solidifying, still envelops the top portions of the microprojections and forms a bridge of continuous drug coating material between them. The bulk of the drug coating material is held between the microprojections in this embodiment.

The convergence of the top portions of the microprojections in a group further functions to protect the drug coating from being pushed off the top portions of the microprojections because much of the drug coating is, for example, under the pinnacle formed by the tips of the microprojections and therefore shielded by the tips of the microprojections during penetration of the stratum corneum. In an embodiment in which the top portions of microprojections in a group are apart sufficiently on top at the tips as well as lower in the shafts of the microprojections, there can be a meniscus on the top of the drug coating as well as in the bottom of the drug coating, similar to what is shown in FIG. 4.

A microprojection array can be made, for example, from a sheet of material by chemical etching. Methods for forming structures that are small (in the range of tens to hundreds of microns) by chemical etching are known in the art. A substrate material, generally flat as a sheet, such as a titanium sheet, can be chemically etched. In generally, a photoresist or a photo-sensitive polymer is laid on a substrate. A pattern is imaged on the photoresist (e.g., with ultra-violet light) and then the photoresist is then developed to provide a patterned polymer layer on the substrate. The patterned polymer layer protects portions of the substrate and leaves other portions unprotected. The substrate with the patterned polymer layer is exposed to an etching liquid, for example, as in a process of spraying the etching liquid on the substrate (with the patterned polymer layer thereon). The part of the substrate that is not protected by the patterned polymer layer is corroded, forming a patterned substrate having microblades that lie flat along the plane of the substrate. The microblades are then cleaned. The microblades are bent using dies. A microblade is bent such that an elongated portion extends normally from the plane of the substrate. This results in a microprojection array on a microprojection member. The microblades are bent using dies. When a microprojection array is made this way, the resulting microprojection array on a microprojection member has the microblades, including the top portions and the bottom portions, and the rest of the base layer are made of the same continuous piece material and is an integral piece.

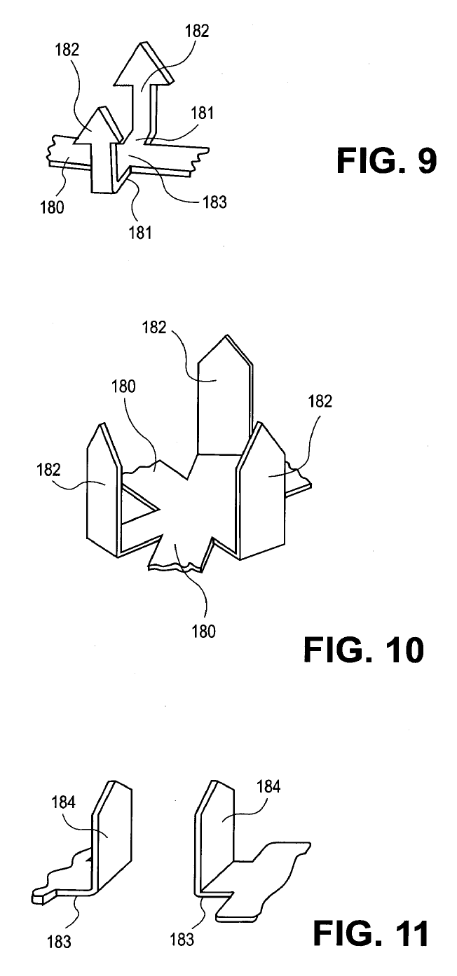

In some embodiments, after a microprojection has been oriented, such as by lifting or bending a portion in the normal (i.e., generally perpendicular) direction, a portion of the microprojection extends along the plane of the substrate (the “planar portion”) to a bend. Past the bend, the normally extending portion projects upward from the plane of the substrate with the other microprojections, preferably in a regular pattern of repeated units of microprojections, to form the microprojection array. In certain designs, such as shown in FIG. 9, the planar portions 181 of a group (e.g., a pair) of microprojections extend outward from one another (in a radiating form), although the top (distal) portions of the microprojections may converge or extend in parallel. Such a design can be achieved, for example, by forming the microprojections in the group about a common area 183 of substrate material. The microprojections 182 are supported on the connecting branches 180, which connect between the groups on the microprojection member.

It is noted that a group can also contain more than two microprojections. For example, as shown schematically in FIG. 10, there can be three microprojections 182 in a group. Such a group can extend from a common area 183, for example, with connecting branches 180 in a honeycomb design supporting the microprojections 182.

In other designs, the planar portions 183 of a group of microprojections extend toward one another (as opposite from a radiating form) whereas the top portions extend in parallel or converge out of the plane of the microprojection member, as shown in FIG. 11.

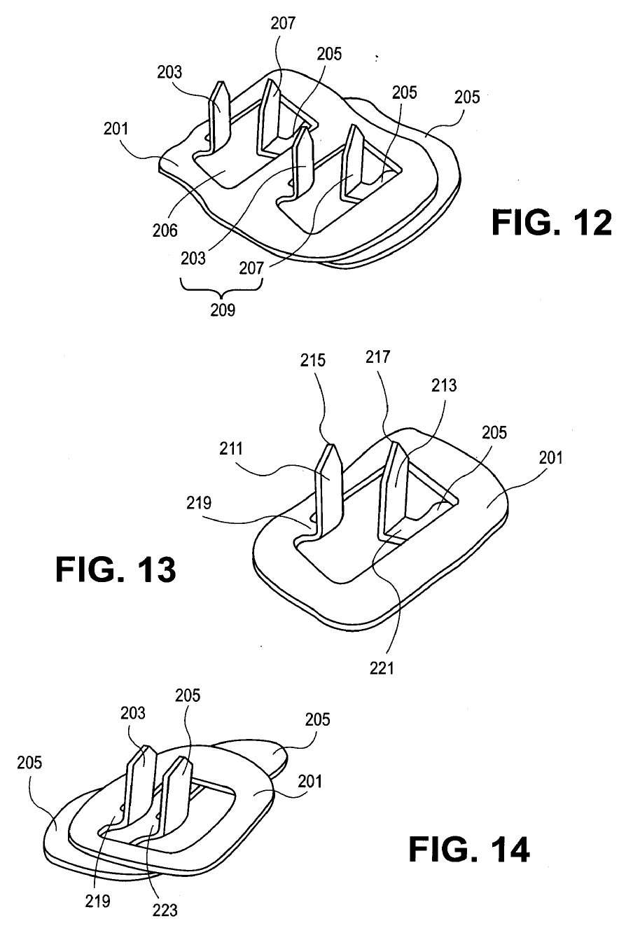

Another way to make a microprojection array with groups of microprojections is, for example, by stacking two layers of microprojections together so the microprojections of one layer protrude through openings of the other layer. As shown in FIG. 12, a top microprojection base layer (or simply “top microprojection layer”) 201 has top microprojections 203 extending out of the plane of the top microprojection layer 201. On the top microprojection layer 201 are a plurality of top openings 206. The top microprojection 203 is positioned near the edge of the top openings 206. A bottom microprojection base layer (or simply “bottom microprojection layer”) 205 is situated under the top microprojection layer 201. A plurality of bottom microprojections 207 arising from the bottom microprojection layer 201 extend through the top openings 206 near the top microprojections 203 to form groups 209 of microprojections.

FIG. 13 shows the embodiment of FIG. 12 in more detail. In the embodiment of FIG. 12 and FIG. 13, the microprojections in the top microprojection layer 201 have a shorter distally (i.e., upwardly in the figure) extending top portion 211 than the distally extending top portions 213 of the bottom microprojection layer 205. In this way, the tips 215 of the top microprojections 211 and the tips 217 of the bottom microprojections are about even over the whole microprojection member, which is composed of the top microprojection layer 201 and the bottom microprojection layer 205, including the corresponding microprojections thereon. Alternatively, the distally extending top portions of the top microprojections can have about the same length as the upwardly extending top portions of the bottom microprojections. To prevent relative movement between the top microprojections and the bottom microprojections, the two microprojection layers 201, 205 can be thermally joined together, e.g., by welding or other techniques known in the art. When stacked together so that their microprojections together form a microprojection array, the two or more microprojection layers can be considered as a single microprojection member.

Grouping of microprojections increases drug loading, e.g., by forming drug coating bridges. Further, by increasing the number of microprojections per unit planar area in a microprojection member, the capacity for loading drug is increased. As used herein, unless specified to be otherwise, “planar area” of a microprojection member refers to the overall area of the microprojection member without subtracting off the area of the openings. However, even with the area of the openings being accounted for, if base layers are stacked so that openings of different layers overlap, drug loading capability per unit exposed area for the microprojection is increased with the present invention compared to prior devices.

In the embodiment shown in FIG. 12 and FIG. 13, in a group of microprojections, a planar portion (extending along the plane of the base layer, e.g., 219, 221) of microprojection (e.g., 203, 207) from each microprojection layer (e.g., top layer and bottom layer) points toward the microprojection (e.g., 207, 203) of the other layer. The planar portions and the top portions of the microprojections were formed by bending or lifting the top portion of the microprojections from the plane of the sheet material after etching. Of course, another alternative, as shown in FIG. 14, is to have the two microprojection layers 201, 205 stacked together such that in a group one planar portion 219 of microprojection of a first layer 201 points toward a planar portion 223 of microprojection of a second layer 205 while the microprojection planar portion 223 from the second layer 205 points away from the microprojection planar portion 219 of the first layer 201.

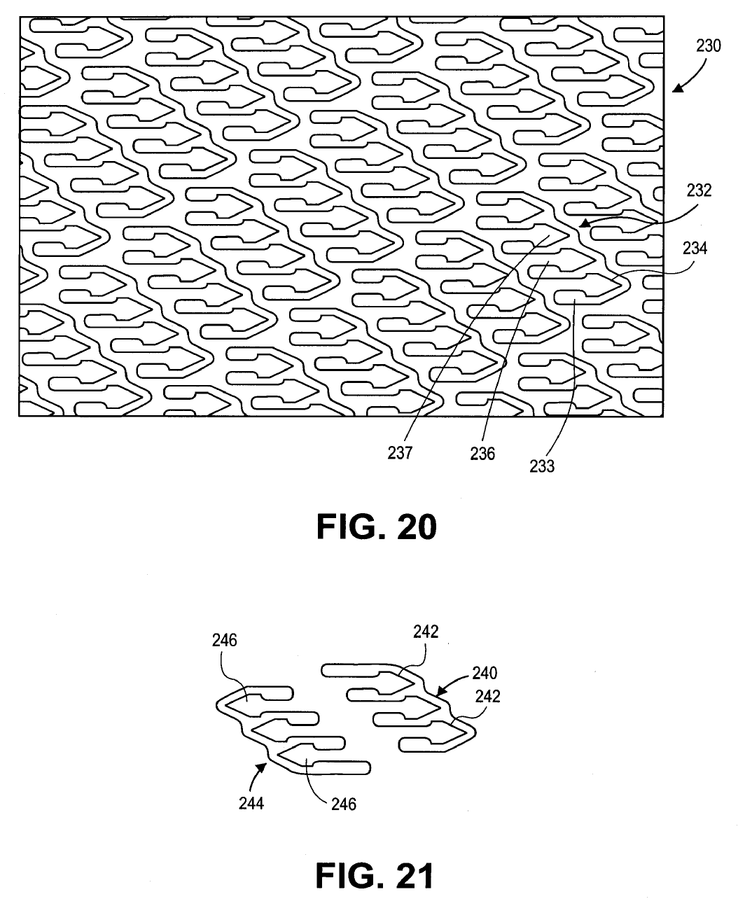

FIG. 20 illustrates a plan view in portion of yet another embodiment of a design of groups of microprojections according to the present invention. In FIG. 20, to better illustrate the positions of the microprojections, the microprojections are shown to be not yet bent or lifted to project an angle from the plane of the substrate (or base layer). In this design, from a single substrate, a microprojection layer (shown only in portion) 230 can be formed to have cells 232 including opening 233 and multiple (here four) microblades 234 all extending in the same planar direction before the microblades 234 are bent to angle from the plane of the substrate. The microblades 234 are offset so the neck 236 of a microblade is adjacent to the arrowhead 237 of another microblade. In this embodiment, all the microblades in all the cells are pointed at the same planar direction before the microblades are bent. In a specific example, a microprojection layer can be made from a substrate with an array of about 1500 microprojections/cm² (in a specific embodiment: exactly 1392 microprojections/cm²), which is substantially higher in density than designs with one or two microblades per cell.

Of course, a cell can have one, two, three, four, or more microblades. Cells have two or more microblades, preferably three or more microblades in certain applications designs, e.g., where not many layers are stacked together. Cells with multiple microblades can be called “supercells.” Designs with supercells can substantially increase the amount of drug that can be coated on the microblades. Microprojection layers with supercells can be used in stacking and drug coating similar to other microprojection layers without supercells.

It is understood that another design is that in some of the cells the microblades point at a different direction from other cells. For example, as shown in FIG. 21, which shows a microprojection layer design in portion, a first cell 240 has microblades 242 pointing to one direction and a neighboring second cell 244 has microblades 246 pointing to a direction opposite to that of the microblades 242 of the first cell 240. In FIG. 21, the first cell 240 and second cell 244 are offset in the Y direction of the Cartesian coordinate. Thus, a microblade 242 of the first cell, although is parallel to an adjacent microblade 246, does not extend on (or lie on) the same line therewith. When the microblades are bent to angle from the plane of the substrate, the microprojections resulting from microblades 242 from the first cell 240 can face squarely microblades 246 of second cell 244. However, the microblades 242 and microblades 246 may be designed to face not exactly face-to-face but somewhat obliquely or slightly offset. In this case, obliquely facing is still considered to be “facing.”

Furthermore, the microblade positions can be designed such that a first cell (similar to first cell 240 of FIG. 21) is not offset from a second cell (similar to the second cell 244 of FIG. 21) in the offset way of FIG. 21, but rather are such that a microblade in the first cell and a microblade in the second cell line along the same line.

Further, it is understood that in the same cell, microblades can that extend in different directions can be designed. For example, in the same cell, two adjacent microblades can be pointing at opposite direction but the arrowheads are staggered or offset or both so that the arrowhead of a microblade is adjacent to the neck of an adjacent microblade (if viewed before the microblades are bent) in the cell. There can be two, three, or more microblades that extend in different directions in a cell.

To increase drug loading, one or more depressions can be formed on the surface of the face of the microblades. The depressions can have a variety of shapes, such as round, oval, polygonal, elongated, star-shaped, and the like. A preferred shape is an elongated channel formed along the shaft of the microprojection, e.g., along the top portion of the microprojection. Further, the microprojection can have a depression on each of the two faces of the microblade. The depressions can extend through the microblade forming a throughhole. The depression can be on a face of the microprojection facing the other microprojection in the group or it can be on the face facing away from the microprojection in the group. In some embodiments, depressions can be located on one microprojection or on multiple microprojections in the group. Thus, the microprojections can increase the drug loading by providing more surface area on the microprojections and by providing a large volume between the microprojections.

In another alternative one face of a microblade can be sculptured to have a depression, such as a channel, and the other face can have a more rounded, or bowed surface akin to a portion of an annular convex surface. For example, the microblade can have an elongated channel on one face and a bowed elongated back on the opposite face. In this way, the microblade has a top portion that is generally thumbnail shape.

The top portion, including the tip, of a microprojection can also have a variety of shapes. For example, the top portion can have an arrowhead shape (e.g., as shown in FIG. 9), a half-arrowhead shape (like that shown in FIG. 2), a tombstone shape with a wedge-shaped top (as shown in FIG. 11), a rounded top, a flat top, and the like.

The drug coating can include one or more of a variety of drugs or biologically active agents. Such drugs include traditional pharmaceuticals, as well as small molecules and biologics. Examples of such drugs or biologically active agents include, without limitation, leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotrophin (ACTH), ACTH analogs such as ACTH (1-24), calcitonin, vasopressin, deamino[Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotrop ins (urofollitropin (F SH) and LH), oxytocin, streptokinase, tissue plasminogen activator, urokinase, ANP, ANP clearance inhibitors, BNP, VEGF, angiotensin II antagonists, antidiuretic hormone agonists, bradykinin antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and mixtures thereof.

The drugs or biologically active agents can also be in various forms, such as free bases, acids, charged or uncharged molecules, components of molecular complexes or nonirritating, pharmacologically acceptable salts. Further, simple derivatives of the active agents (such as ethers, esters, amides, etc.), which are easily hydrolyzed at body pH, enzymes, etc., can be employed.

The drugs or biologically active agents can be incorporated into a liquid drug coating material and coated onto the microprojections.

Typically, the drug or biologically active agent is present in the drug coating formulation at a concentration in the range of approximately 0.1-30 wt %, preferably 1-30 wt %.

Preferably, the amount of drug contained in the biocompatible coating (i.e., dose) is in the range of approximately 1 μg-1000 μg, more preferably, in the range of approximately 10-200 μg per dosage unit. Even more preferably, the amount of the drug contained in the biocompatible coating is in the range of approximately 10-100 μg per dosage unit.

Preferably, the pH of the coating formulation is adjusted to provide conditions for maintaining the stability of the drug selected for incorporation in the drug coating formulation. In certain embodiments of the invention, the viscosity of the coating formulation is enhanced by adding low volatility counterions. In certain embodiments, the drug has a positive charge at the formulation pH and the viscosity-enhancing counterion comprises an acid having at least two acidic pKas. Suitable acids include, without limitation, maleic acid, malic acid, malonic acid, tartaric acid, adipic acid, citraconic acid, fumaric acid, glutaric acid, itaconic acid, meglutol, mesaconic acid, succinic acid, citramalic acid, tartronic acid, citric acid, tricarballylic acid, ethylenediaminetetraacetic acid, aspartic acid, glutamic acid, carbonic acid, sulfuric acid and phosphoric acid.

In some embodiments of the invention, the amount of counterion is preferably sufficient to neutralize the charge of the drug. In such embodiments, the counterion or the mixture of counterion is preferably sufficient to neutralize the charge present on the agent at the pH of the formulation. In additional embodiments, excess counterion (as the free acid or as a salt) is added to the drug to control pH and provide adequate buffering capacity.

In one embodiment, the counterion comprises a viscosity-enhancing mixture of counterions chosen from the group consisting of citric acid, tartaric acid, malic acid, hydrochloric acid, glycolic acid and acetic acid. Preferably, the counterions are added to the formulation to achieve desired viscosity.

The viscosity of the drug coating formulation in liquid form is affected by the nature of the polymeric material and counterions present. The drug coating formulations typically have a viscosity of less than approximately 500 centipoise (typically measured at 25° C. and at a shear strain rate of 100/sec) and greater than 3 centipoise (cp), preferably a viscosity in the range of about 20-200 cp. Such viscosity ranges are suitable for forming a drug coating on the microprojections, for example, wherein capillary force can hold the liquid drug coating formation between the microprojections in a group until the formulation is solidified.

In certain embodiments, the viscosity-enhancing counterion contains an acidic counterion, such as a low volatility weak acid. Preferably, the low volatility weak acid counterion exhibits at least one acidic pKa and a melting point higher than about 50° C. or a boiling point higher than about 170° C. at atmospheric pressure. Examples of such acids include, without limitation, citric acid, succinic acid, glycolic acid, gluconic acid, glucuronic acid, lactic acid, malic acid, pyruvic acid, tartaric acid, tartronic acid and fumaric acid.

In another embodiment, the counterion comprises a strong acid. Preferably, the strong acid exhibits at least one pKa lower than about 2. Examples of such acids include, without limitation, hydrochloric acid, hydrobromic acid, nitric acid, sulfonic acid, sulfuric acid, maleic acid, phosphoric acid, benzene sulfonic acid and methane sulfonic acid. Another embodiment is directed to a mixture of counterions, wherein at least one of the counterion comprises a strong acid and at least one of the counterions comprises a low volatility weak acid.

Another preferred embodiment is directed to a mixture of counterions, wherein at least one of the counterions comprises a strong acid and at least one of the counterions comprises a weak acid with high volatility. Preferably, the volatile weak acid counterion exhibits at least one pKa higher than about 2 and a melting point lower than about 50° C. or a boiling point lower than about 170° C. at atmospheric pressure. Examples of such acids include, without limitation, acetic acid, propionic acid, pentanoic acid and the like.

The acidic counterion is preferably present in an amount sufficient to neutralize the positive charge present on the drug at the pH of the formulation. In additional embodiments, excess counterion (as the free acid or as a salt) is added to control pH and to provide adequate buffering capacity.

In another embodiment of the invention, the coating formulation includes at least one buffer. Examples of such buffers include, without limitation, ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine and mixtures thereof.

In one embodiment of the invention, the coating formulation includes at least one antioxidant, which can be sequestering agents, such sodium citrate, citric acid, EDTA (ethylene-dinitrilo-tetraacetic acid) or free radical scavengers such as ascorbic acid, methionine, sodium ascorbate and the like. Presently preferred antioxidants comprise EDTA and methionine.

In the noted embodiments of the invention, the concentration of the antioxidant is in the range of approximately 0.01-20 wt. % of the coating formulation. Preferably the antioxidant is in the range of approximately 0.03-10 wt. % of the coating formulation.

In one embodiment of the invention, the coating formulation includes at least one surfactant, which can be zwitterionic, amphoteric, cationic, anionic, or nonionic, including, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives, such as sorbitan laurate, alkoxylated alcohols, such as laureth-4 and polyoxyethylene castor oil derivatives, such as CREMOPHOR EL.

In one embodiment of the invention, the concentration of the surfactant is in the range of approximately 0.01-20 wt % of the coating formulation. Preferably the surfactant is in the range of approximately 0.05-1 wt % of the coating formulation.

In a further embodiment of the invention, the coating formulation includes at least one polymeric material or polymer that has amphiphilic properties, which can comprise, without limitation, cellulose derivatives, such as hydroxyethylcellulose (HEC), hydroxypropylmethylcell-ulose (HPMC), hydroxypropycellulose (HPC), methylcellulose (MC), hydroxyethylmethylcellulose (HEMC), or ethylhydroxy-ethylcellulose (EHEC), as well as pluronics.

In one embodiment of the invention, the concentration of the polymer presenting amphiphilic properties in the coating formulation is preferably in the range of approximately 0.01-20 wt %, more preferably, in the range of approximately 0.03-10 wt. % of the coating formulation.

In another embodiment, the coating formulation includes a hydrophilic polymer selected from the following group: hydroxyethyl starch, carboxymethyl cellulose and salts of, dextran, poly(vinyl alcohol), poly(ethylene oxide), poly(2-hydroxyethylmethacrylate), poly(n-vinyl pyrolidone), polyethylene glycol and mixtures thereof, and like polymers.

In an embodiment, the concentration of the hydrophilic polymer in the coating formulation is in the range of approximately 1-30 wt %, more preferably, in the range of approximately 1-20 wt % of the coating formulation.

In another embodiment of the invention, the coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose, stachyose, mannitol, and other sugar alcohols.

Preferably, the concentration of the biocompatible carrier in the coating formulation is in the range of approximately 2-70 wt %, more preferably, in the range of approximately 5-50 wt % of the coating formulation.

In another embodiment, the coating formulation includes a stabilizing agent, which can comprise, without limitation, a non-reducing sugar, a polysaccharide or a reducing sugar.

Suitable non-reducing sugars for use in the methods and compositions of the invention include, for example, sucrose, trehalose, stachyose, or raffinose.

Suitable polysaccharides for use in the methods and compositions of the invention include, for example, dextran, soluble starch, dextrin, and insulin.

Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose, and the like.

Preferably, the concentration of the stabilizing agent in the coating formulation is at ratio of approximately 0.1-2.0:1 with respect to the drug, more preferably, approximately 0.25-1.0:1 with respect to the drug.

In another embodiment, the coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline. The concentration of the vasoconstrictor, if employed, is preferably in the range of approximately 0.1 wt % to 10 wt % of the coating formulation.

In another embodiment of the invention, the coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In yet another embodiment of the invention, the coating formulation includes a solubilising/complexing agent, which can comprise Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, rnaltosyl-beta-Cyclodextrin, hydroxypropyl beta-Cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-Cyclodextrin, sulfobutylether-beta-Cyclodextrin, and sulfobutylether-gamma-Cyclodextrin. Most preferred solubilising/complexing agents are beta-Cyclodextrin, hydroxypropyl beta-Cyclodextrin, 2-hydroxypropyl-beta-Cyclodextrin and sulfobutylether7 beta-Cyclodextrin. The concentration of the solubilising/complexing agent, if employed, is preferably in the range of approximately 1 wt. % to 20 wt. % of the coating formulation.

In another embodiment of the invention, the coating formulation includes at least one non-aqueous solvent, such as ethanol, isopropanol, methanol, propanol, butanol, propylene glycol, dimethysulfoxide, glycerin, N,N-dimethylformamide and polyethylene glycol 400. Preferably, the non-aqueous solvent is present in the coating formulation in the range of approximately 1 wt % to 50 wt % of the coating formulation. Other known formulation adjuvants can also be added to the coating formulations provided they do not adversely affect the necessary solubility and viscosity characteristics of the coating formulation and the physical integrity of the dried coating.

In one embodiment of the invention, the thickness of the biocompatible coating (drug coating) is less than 25μ, more preferably, less than 10μ, as measured from the microprojection surface. The desired coating thickness is dependent upon several factors, including the required dosage and, hence, coating thickness necessary to deliver the dosage, the density of the microprojections per unit area of the sheet, the viscosity and concentration of the coating composition and the coating method chosen. In accordance with one embodiment of the invention, the method for delivering a drug contained in the biocompatible coating on the microprojection member includes the following steps: the coated microprojection member is initially applied to the patient's skin via an actuator, wherein the microprojections pierce the stratum corneum. The coated microprojection member is preferably left on the skin for a period lasting from 5 seconds to 24 hours. Following the desired wearing time, the microprojection member is removed.

The drug coating can be formed on microprojections by using rollers, for example, with the method and apparatus described by US patent publication 20020132054, which in incorporated by reference herein in its entirety. Briefly described, a coating liquid containing a drug is conveyed to a liquid holding surface having a coating transfer region, such as a surface of a rotating drum. A microprojection member having a microprojection array is passed over the coating transfer region such that the microprojections dip their top portions into the coating liquid at the desired depth. The depth of the coating liquid at the coating transfer region is controlled so that right amount of drug coating liquid is deposited on the microprojection at the right height on the microprojection. The depth of the coating liquid at the coating transfer region can be controlled, for example, by using a doctor blade.

After a liquid drug coating has been deposited on the microprojections, the liquid drug coating is dried to solidify the liquid drug coating. The drying can be done at ambient (room) conditions. Further, various drying techniques can be used, such as using heat, controlled lower vapor pressure of the solvent in atmosphere above the liquid, etc.

The microprojection array can be applied on the skin of an individual, for example, by using an applicator, as done with other conventional microprojection arrays.

### EXAMPLES

Below are examples of specific embodiments for carrying out the present invention. The examples are offered for illustrative purposes only, and are not intended to limit the scope of the present invention in any way.

### Example 1

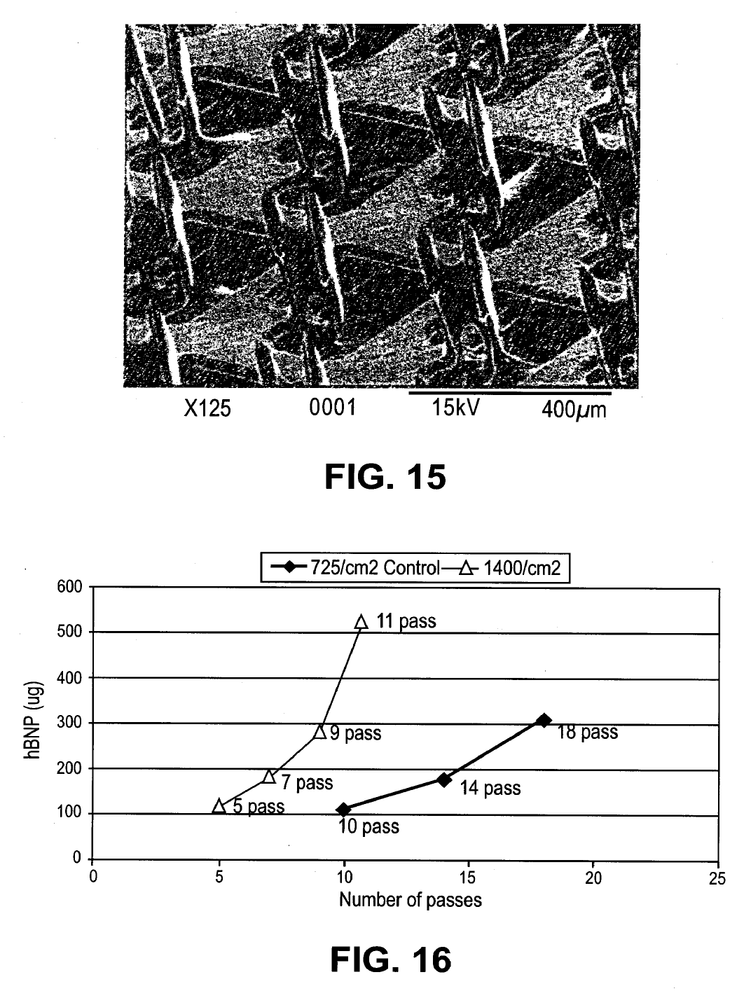

FIG. 15 shows a photograph of an microprojection array having microprojection pairs with drug coating, made by stacking two layers of microprojections together wherein the microprojections of the bottom base layer protrude through the window openings in the top microprojection base layer. The microprojection member was made by chemically etching a titanium substrate to obtain microblade arrays 2 cm² in size and 25μ thick with methods known in the art to form arrowheaded microblades and stacking two microblade arrays to form a microprojection member.

A first substrate titanium sheet a little thicker than 25μ was coated with photoresist, imaged for a pattern to form microblades and chemically etched with an etching solutions, such as ferric chloride solution, known in the art. The patterned polymer layer protected portions of the substrate and left other portions unprotected. After etching, the part of the substrate that was not protected by the patterned polymer layer was corroded, forming a patterned substrate having microblades that lay flat along the plane of the substrate. The microblades were then cleaned and bent using dies. This resulted in a perpendicularly extending top portion of about 225μ length, 116μ width, 25μ thickness. This formed the top microprojection layer with a microblade array (the first microblade array). The top microprojection layer had a microproblade density of 725/cm². A microblade in the top microprojection layer had a planar surface area of about 5.8×10⁻³ mm². In a similar way, a bottom microprojection layer was formed to result in microproblades (microprojections) with perpendicularly extending top portion of about 250μ length, 116μ width, and 25μ thickness. This formed the bottom microprojection layer with a microblade array (the second microblade array). In this way, when stacked to pair the microblades, the microblade from the bottom layer would match the microblade from the top layer at their tips. The patterns of the two layers were designed such that the windows of the two layers about coincided when the microprojections of the bottom layer protruded through the windows of the top layer matching with the top microprojections with an offset gap of about 40μ within a pair of matched microprojections in the fashion of FIG. 14. As can be seen in FIG. 15, the planar portions associated with the microblades in a pair extended along the plane of the microprojection layers in the same direction. The edges of the two layers were aligned and affixed together by thermal fusion (welding).

The top portions of the microprojections in the microprojection member were coated with a drug formulation by dip coating with multiple passes and dried so that the liquid drug formulation solidified, using standard dip coating method known in the art, see U.S. Pat. No. 6,855,372 entitled “Method for Coating Skin Piercing Microprojections”. A drug coating known in the art can be used, e.g., those disclosed in US Patent Publications 20020132054, 20050256045. (For example, US Patent Publication 20020132054 discloses drug coatings with human growth hormone and US Patent Publication 20050256045 discloses drug coatings with parathyroid hormone.) Meniscus was seen on the bottom and on the top of the drug coating held between the microblades in the pair.

### Example 2

A first microprojection member with a single base layer was made with the method of Example 1, similar to the top microblade array of Example 1. A second microprojection member with two base layers was made in the fashion of FIG. 15, similar to the double layered microprojection member with two microblade arrays stacked in Example 1. In the second microprojection member, the microblades (microprojections) of the bottom layer protruded through the top layer and paired with corresponding microblades (microprojections) of the top layer. The top microblade array had a microblade (microprojection) density of about 725/cm². The microblades of the top layer had a perpendicularly extending top portion of 225μ length 116μ width 25μ thickness, and a planar surface area of about 5.8×10⁻³ mm². The bottom layer of microblades had a perpendicularly extending top portion of about 250μ length, 116μ width, 25μ thickness, and a planar surface area of about 5.8×10⁻³ mm². When stacked together, the tips of the microblades from the bottom layer and from the top layers are about even in distance from the layers. The two-layered microprojection member had a microprojection density of about 1400/cm². The gap between the microprojections in a pair was about 100μ. The microprojections from the first single layered microprojection member and from the second (double layered) microprojection member were each coated with a coating formulation of the drug hBNP (human brain-type Natriuretic peptide, NATRECOR made by Scios) with 25% hBNP (w/w), 6.25% sucrose (w/w), 0.10% polysorbate 20 (w/w) using standard dip coating method known in the art. The dip coating was done with multiple passes. The process was repeated so that samples with different number of dip coatings were analyzed for drug content on the microprojections. The drug coatings were analyzed by HPLC. FIG. 16 is a graph showing the drug content of the two microprojection members (one double layered and one single layered) of equal overall microprojection member planar surface after a number of passes in dip coating. The curve on the right with the diamond shaped data points symbols the data for the singled layer microprojection members having 725 microprojections/cm². The curve on the left with triangular data symbols shows the data for the two layered microprojection members with 1400 microprojections/cm². The graph shows that the microprojection member with two layers stacked together had substantially higher drug content than the microprojection member with a single layer. In fact, the drug content of the two-layered microprojection member was more than double that of the single layered microprojection member for the same number of passes due to the presence of drug coating bridges between the microprojections in the pairs.

### Example 3

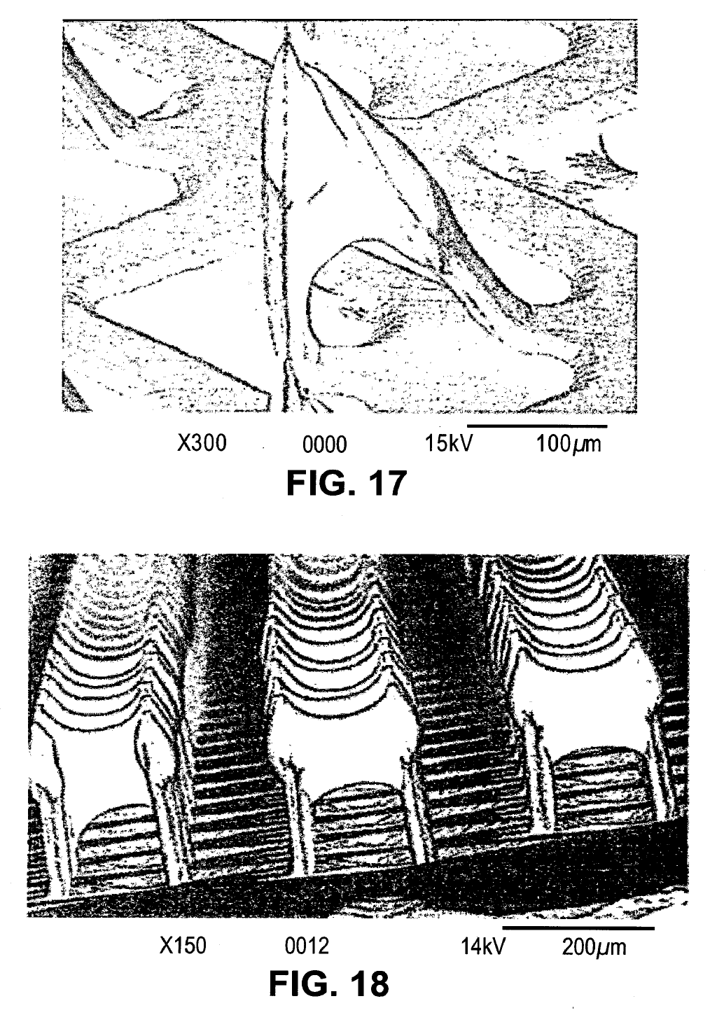

A microprojection member was made from two microprojection layers that were stacked together with a method similar to that of Example 1. The microprojections of the bottom layer protruded through the top layer and paired with corresponding microprojections of the top layer. In a pair, the top layer of microprojections extended from the plane of the array at 50 degrees and leaned towards the microprojection from the bottom layer. The microprojection from the top layer had a top portion of about 225μ length, 116μ width, 25μ thickness, and a planar surface area of about 5.8×10⁻³ mm². The bottom layer of microprojections had a top portion (extending from the plane of the base layer at an angle) of about 225μ length, 116μ width, 25μ thickness, and a planar surface area of about 5.8×10⁻³ mm². The combined arrays formed a pinnacle shape that contained the drug formulation (72.5% w/w granisetron, 27.0% w/w citric acid and 0.44% w/w polysorbate 20). FIG. 17 is an electronmicrograph of a portion of the microprojection member showing such a pinnacle formed by a pair of microprojections. The two-layered microprojection member had a microprojection density of about 725/cm² pinnacles per cm². The gap between the microprojections in a pair was about 140μ.

### Example 4

A first microblade array with a single base layer and a second microblade array with a single base layer were made with the method similar to Example 1 except that the microblade arrays were designed to stack in the manner of FIG. 13 with a gap offset separating the microblades in a pair of 100μ. In this embodiment, the planar portions of the microblades in a pair extended towards each other. A drug coating was coated on the microprojections with coating method similar to the above examples. FIG. 18 shows an electronmicrograph of the microprojection pairs after a drug coating was formed thereon. Meniscus was seen on the bottom and on the top of the drug coating held between the microblades in each of the pairs.

### Example 5

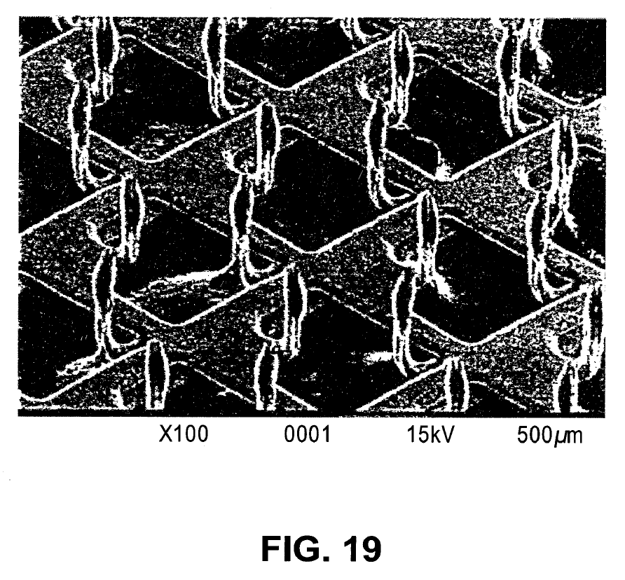

A first microblade array with a single base layer and a second microblade array were made with the method similar to Example 4 except that the microblade arrays were designed to stack in the manner of FIG. 13 with a gap offset separating the microblades in a pair of about 250μ and such that after drug coating the microblades in a pair each had its own drug coating. There was no drug coating that continued and bridged between the top perpendicular portions of the microblades. However, this design would still facilitate better penetration into the stratum-corneum because the close proximity of the microblades would help to hold taut the skin between a pair just prior to penetration. FIG. 19 shows an electronmicrograph of the microprojection pairs with drug coating.

The entire disclosure of each patent, patent application, and publication cited or described in this document is hereby incorporated herein by reference. The practice of the present invention will employ, unless otherwise indicated, conventional methods used by those in pharmaceutical product development within those of skill of the art. Embodiments of the present invention have been described with specificity. The embodiments are intended to be illustrative in all respects, rather than restrictive, of the present invention. It is to be understood that various combinations and permutations of various constituents, parts and components of the schemes disclosed herein can be implemented by one skilled in the art without departing from the scope of the present invention.

What is claimed is:

1. An apparatus for stratum-corneum piercing drug delivery, comprising: a microprojection member having a plurality of stratum-corneum piercing microprojections for piercing stratum-corneum to facilitate drug delivery, wherein at least some of the microprojections are arranged in groups, each group having at least two adjacent microprojections.

2. The apparatus of claim 1, wherein at least some of the microprojections are blade shaped.

3. The apparatus of claim 2, wherein the blade shaped microprojections have a sharp cutting point.

4. The apparatus of claim 2 wherein in at least some of the groups the microprojections have a face facing other microprojections in the group, and further comprise a drug coating on at least a portion of the microprojections in the group.

5. The apparatus of claim 2 wherein in a group at least one microprojection projects at an angle to lean toward another microprojection in the group.

6. The apparatus of claim 2 wherein in at least some of the groups the microprojections are together in pairs and wherein the microprojections in a pair have top portions that are substantially parallel relative to each other.

7. The apparatus of claim 2 wherein each microprojection has a base and wherein in at least some of the groups the microprojections are together in pairs and in a pair the microprojections are spaced apart at the base by less than 200 microns.

8. The apparatus of claim 2 wherein each microprojection has a base and wherein in at least some of the groups of microprojections, the microprojections are together in pairs and in a pair the microprojections are spaced apart at the base by 10 microns to 100 microns.

9. The apparatus of claim 2 wherein in at least some of the groups the microprojections have a drug coating that coats the microprojections of the group as a continuous coating.

10. The apparatus of claim 2 wherein each microprojection has a tip and wherein in at least some of the groups of microprojections, the microprojections are grouped in pairs and wherein a drug coating coats a pair of microprojections as a continuous coating near the tips of the microprojections in the pair.

11. The apparatus of claim 2 wherein each microprojection has a top portion extending out of a plane of the microprojection member and in at least some of the groups the microprojections have base portions extending more along a plane of the microprojection member than the top portions do and towards other microprojections portions in the group.

12. The apparatus of claim 2 wherein each microprojection has a top portion extending out of a plane of the microprojection member and in at least some of the groups the microprojections have base portions extending more along a plane of the microprojection member and away from other microprojections in the group than the top portions do.

13. The apparatus of claim 2 wherein in at least some of the groups the microprojections have shafts.

14. The apparatus of claim 13, wherein the shafts are of different lengths.

15. The apparatus of claim 2 wherein in at least some of the groups the microprojections form a pair of microprojections having shafts of different lengths.

16. The apparatus of claim 2 wherein in at least some of the groups the microprojections form a pair of microprojections having shafts of different lengths, wherein one shaft of a microprojection leans toward a shaft of another microprojection forming a pinnacle between the two microprojections.

17. The apparatus of claim 2 wherein in at least some of the groups the microprojections are a pair of microprojections having shafts of different lengths, wherein a microprojection with a longer shaft leans toward a microprojection with a shorter shaft to intercept the microprojection with the shorter shaft thereby, forming a pinnacle between the two microprojections.

18. The apparatus of claim 17, wherein the pinnacle formed between two microprojections has an angle of between a 10 degree and a 60 degree angle.

19. The apparatus of claim 2 wherein each microprojection has a base.

20. The apparatus of claim 19 wherein in at least some of the groups the microprojections are grouped together in pairs and the bases of the microprojections in a pair are spaced between 10 microns to 100 microns apart.

21. The apparatus of claim 20 wherein in at least some of the groups a pinnacle is formed between the microprojections, the pinnacle being at an angle between 10 degrees to 60 degrees.

22. The apparatus of claim 2 wherein at least some of the microprojections in a group are from a cell and arising from the same base layer.

23. An apparatus for stratum-corneum piercing drug delivery, comprising: a microprojection member having a plurality of stratum corneum piercing microprojections for piercing stratum corneum to facilitate drug delivery, at least some of the microprojections are arranged in groups of at least two adjacent microprojections; in at least some of the groups the microprojections have shafts of different lengths, a microprojection extending normally from the microprojection member and another microprojection leaning to the shorter microprojection forming a pinnacle; and in said at least some of the groups a continuous drug coating coats at least a top portion of the microprojections in the group, the drug coating having a meniscus bridging the microprojections in the group.

24. A method for stratum-corneum piercing drug delivery to an individual comprising: (a) providing a plurality of stratum corneum piercing microprojections for piercing stratum corneum to facilitate drug delivery, (b) arranging at least some of the microprojections in groups of at least two adjacent microprojections, and (c) piercing the stratum-corneum of said individual with microprojections from the microprojection array.

25. The method of claim 24 comprising providing blade shaped microprojection pairs.

26. The method of claim 25 comprising providing blade shaped microprojections pairs having a face, said face facing other microprojections in the group.

27. The method of claim 25 comprising providing blade shaped microprojection pairs coated with a drug coating on at least a portion of the microprojections.

28. The method of claim 24 comprising providing microprojection pairs with at least one microprojection in a pair projecting at an angle toward another microprojection in the pair.

29. The method of claim 24 comprising providing microprojections having a base.

30. The method of claim 29 comprising providing microprojection pairs in which the microprojections are spaced apart at the base by 10 microns to 100 microns.

31. The method of claim 24 comprising providing microprojection pairs coated with a drug coating on said pair of microprojections, said drug coating forming a continuous coating.

32. The method of claim 24 comprising providing microprojection pairs having shafts of different lengths.

33. A method for forming a stratum-corneum piercing drug delivery apparatus, comprising: (a) forming a microprojection array, the microprojection array having a plurality of stratum corneum piercing microprojections for piercing stratum corneum to facilitate drug delivery, and (b) arranging at least some of the microprojections in groups of at least two adjacent microprojections.

34. The method of claim 33 comprising forming at least some of the microprojections having a face facing other microprojection in the group.

35. The method of claim 33 comprising providing microprojection pairs coated with a drug coating on at least a portion of the microprojections.

36. The method of claim 33 comprising providing microprojection pairs where one microprojection in a pair projects at an angle toward another microprojection in the pair.

37. The method of claim 33 comprising providing microprojection pairs where a first microprojection has a shaft at one length and a second microprojection has a shaft of a length different than the length of the shaft of the first microprojection.

38. The method of claim 33 comprising providing microprojection pairs coated with a continuous drug coating on at least one pair of microprojections.

39. The method of claim 33 comprising providing microprojections having a base.

40. The method of claim 39 comprising providing microprojection pairs where the bases of a pair of microprojections are set apart by less than 200 μm.

41. The method of claim 33 comprising providing a plurality of cells having multiple microprojections from a substrate.
